(19) United States
(12) Patent Application Publication
(10) Pub. No.: US 2017/0000349 A1
(43) Pub. Date: Jan. 5, 2017

(54) METHODS AND SYSTEMS FOR BLOOD GLUCOSE MONITORING

(71) Applicants: Liron HADAR, Haifa (IL); Erez RAPHAEL, Nofit (IL); Shai EISEN, New York, NY (US); Jonathan KANETY, Rehovot (IL); Yoav TIKOCHINSKY, Tel-aviv (IL); Haim KRIEF, Hedera (IL); Daniel DARST, Zimmerman, MN (US); Eric Jason KRAUSE, Big Lake, MN (US)

(72) Inventors: Haim KRIEF, Hedera (IL); Erez RAPHAEL, Nofit (IL)

(21) Appl. No.: 15/125,667

(22) PCT Filed: Mar. 13, 2015
(86) PCT No.: PCT/US15/20545

§ 371 Date: Sep. 13, 2016

Related U.S. Application Data
(60) Provisional application No. 61/952,703, filed on Mar. 13, 2014.

(51) Int. Cl. A61B 5/00; H04W 4/00; H04L 29/08; H04B 5/00; A61B 5/15; G01N 33/49

(52) U.S. Cl. CPC A61B 5/0022; A61B 5/0004; A61B 5/002; A61B 5/150358; A61B 5/6898; G01N 33/49; H04L 67/025; H04L 67/18; H04B 5/0031; H04W 4/00

(57) ABSTRACT

The present invention provides for an apparatus, including: a lancing device; a cartridge; where the lancing device is configured to house the cartridge; where the cartridge is configured to house a plurality of test strips and a glucose monitoring apparatus; where the glucose monitoring apparatus is configured to determine a glucose test result from at least one test strip of the plurality of test strips.

### RELATED APPLICATIONS

This application claims the priority of U.S. provisional application U.S. Patent Appln. No. 61/952,703; filed Mar. 13, 2014; entitled “METHOD AND SYSTEM OF BLOOD GLUCOSE MONITORING,” which is incorporated herein by reference in its entirety for all purposes.

### TECHNICAL FIELD

In some embodiments, the instant invention is related to methods and systems for monitoring blood glucose.

### BACKGROUND

Blood glucose monitoring is used to test the concentration of glucose in the blood (glycemia). Such testing is important in the care of diabetes mellitus. A blood glucose test is performed by piercing the skin to draw blood, then applying the blood to a chemically active disposable ‘test-strip’.

### SUMMARY OF INVENTION

In some embodiments, the instant invention provides for an apparatus, including: a lancing device; a cartridge; where the lancing device is configured to house the cartridge; where the cartridge is configured to house a plurality of test strips and a glucose monitoring apparatus; where the glucose monitoring apparatus includes: a connecting plug configured to mate with an audio jack phone port, where the audio jack phone port has at least three audio pins; a first ring, where the first ring is positioned on the connecting plug, and where the first ring is configured to mate with a first audio pin of the audio jack phone port; a second ring, where the second ring is positioned on the connecting plug, and where the second ring is configured to mate with a second audio pin of the audio jack phone port; and a third ring, where the third ring is positioned on the connecting plug, and where the third ring is configured to mate with a third audio pin of the audio jack phone port; where at least one of the first, second, and third rings of the connecting plug is configured to receive power for the glucose monitoring apparatus from the audio jack phone port, where at least one of the first, second, and third rings of the connecting plug is configured to receive data from the audio jack phone port, where at least one of the first, second, and third rings of the connecting is configured to transmit data from the glucose monitoring apparatus to the audio jack phone port; and where the glucose monitoring apparatus is configured to determine a glucose test result from at least one test strip of the plurality of test strips. In some embodiments, the apparatus is configured to house at least one test strip. In some embodiments, the apparatus further houses a plurality of test strips. In some embodiments, the apparatus is configured to deploy a lancet of the plurality of lancets and automatically return the lancet of the plurality of lancets into the housing. In some embodiments, the apparatus further includes a button configured to release the lancet. In some embodiments, the apparatus further includes a test strip connector configured to read the test strip glucose level. In some embodiments, the lancing device is configured to eject a lancet. In some embodiments, the apparatus further includes a depth indicator dial configured to allow for adjustment of a lancet penetration depth. In some embodiments, the apparatus further includes a first housing cover configured to cover the disposable lancet. In some embodiments, the apparatus further includes a second housing cover configured to attach to the lancing device at a test strip end position.

In some embodiments, the instant invention provides for a computer system, including: at least one server having software stored on a non-transient computer readable medium; where, upon execution of the software, the at least one server is at least configured to receive glucose test data from a plurality of apparatuses, where each apparatus includes: a lancing device; a cartridge; where the lancing device is configured to house the cartridge; where the cartridge is configured to house a plurality of test strips and a glucose monitoring apparatus; where the glucose monitoring apparatus includes: a connecting plug configured to mate with an audio jack phone port, where the audio jack phone port has at least three audio pins; a first ring, where the first ring is positioned on the connecting plug, and where the first ring is configured to mate with a first audio pin of the audio jack phone port; a second ring, where the second ring is positioned on the connecting plug, and where the second ring is configured to mate with a second audio pin of the audio jack phone port; and a third ring, where the third ring is positioned on the connecting plug, and where the third ring is configured to mate with a third audio pin of the audio jack phone port; where at least one of the first, second, and third rings of the connecting plug is configured to receive power for the glucose monitoring apparatus from the audio jack phone port, where at least one of the first, second, and third rings of the connecting plug is configured to receive data from the audio jack phone port, where at least one of the first, second, and third rings of the connecting is configured to transmit data from the glucose monitoring apparatus to the audio jack phone port; and where the glucose monitoring apparatus is configured to determine a glucose test result from at least one test strip of the plurality of test strips; where the glucose data is delivered to at least one third party user by use of a graphical user interface caused to be displayed by the software. In some embodiments, the at least one third party user is a medical professional.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention. Further, some features may be exaggerated to show details of particular components.

The figures constitute a part of this specification and include illustrative embodiments of the present invention and illustrate various objects and features thereof. Further, the figures are not necessarily to scale, some features may be exaggerated to show details of particular components. In addition, any measurements, specifications and the like shown in the figures are intended to be illustrative, and not restrictive. Therefore, specific structural and functional details disclosed herein are not to be interpreted as limiting, but merely as a representative basis for teaching one skilled in the art to variously employ the present invention.

FIGS. 1A and 1B illustrate an embodiment of the test strip cartridge of the present invention.

FIG. 2 illustrates an embodiment of the test strip cartridge housed in the lancing device of the present invention.

FIGS. 3A and 3B illustrate an embodiment the apparatus of the present invention, where the lancet being inserted into a lancing device of the present invention.

FIG. 4 illustrates an embodiment of the present invention, where the lancet is placed in the lancing device.

FIG. 5 illustrates an embodiment of the present invention, where the Lancet is released into the lancing device.

FIG. 6 illustrates an embodiment of the Blood Glucose Monitor (BGM) (Dongle) of the present invention including a strip port, a glucose meter, and an audio plug connector.

FIG. 7 illustrates an embodiment of the apparatus of the present invention, showing a BGM reading and connecting a test strip to a smart mobile device.

FIG. 8 illustrates an embodiment of a block diagram of the BGM (dongle) of the present invention.

FIG. 9 illustrates an embodiment the apparatus of the present invention, showing a glucose meter audio plug of the BGM.

FIGS. 10 and 11 illustrate some embodiments of the inventive system of the present invention, showing users with diabetes connecting over network servers in accordance with the invention.

FIGS. 12-13 illustrate further aspects of some embodiments of the system of the present invention.

FIG. 14 illustrates an embodiment of the closed apparatus of the present invention.

FIGS. 15A and 15B illustrate embodiments of the components of the apparatus of the present invention.

FIGS. 16A and 16B show exemplary embodiments of the apparatus of the present invention, showing insertion of the test strip cartridge.

FIGS. 17A and 17B show exemplary embodiments of the apparatus of the present invention, showing insertion of a lancet.

FIGS. 18A and 18B show exemplary embodiments of the apparatus of the present invention, showing setting the lancet depth.

FIGS. 19A and 19B show exemplary embodiments of the apparatus of the present invention, showing how a user can perform a control solution test.

FIGS. 20A and 20B show exemplary embodiments of the apparatus of the present invention, showing the removal of a test strip from the cartridge.

FIGS. 21A-21E show exemplary embodiments of the apparatus of the present invention, showing the method of removing the glucose meter from the housing.

FIGS. 22A-22F show exemplary embodiments of the apparatus of the present invention, showing a method for removing a test strip from the cartridge.

FIGS. 23A-23F show exemplary embodiments of the apparatus of the present invention, showing the user performing the glucose test.

### DESCRIPTION OF EXEMPLARY EMBODIMENTS

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator, and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of“in” includes “in” and “on.”

In some embodiments, the present invention is a Blood Glucose Monitoring System (BGMS) for measuring blood glucose levels from at least one fresh capillary whole blood sample. In some embodiments, the whole blood sample can be taken from anywhere on a body, e.g., from a fingertip. In some embodiments, the present invention is a BGMS comprising a blood glucose meter (BGM). An example of a BGM is disclosed in U.S. Pat. No. 8,797,180, and is hereby incorporated by reference in its entirety. In some embodiments, the present invention is a dongle. In some embodiments, the present invention is a dongle configured to connect to a smart mobile device by an audio jack. In some embodiments, the present invention further comprises a plurality of disposable test strips, a strip dispenser cartridge, a lancing device, at least one lancet (e.g., 1, 2, 3, 4, 5, 6, 7, 8, etc.), or any combination thereof. In some embodiments, includes an application configured to be used through software on a smart mobile device. In some embodiments, the present invention is an application configured to communicate with a BGMS and a user. In some embodiments, the system of the present invention is configured to communicate with a plurality of users (e.g., 1, up to 10, up to 10,000, up to 100,000, up to 1,000,000, etc. user(s)) and a third party (e.g., 1, up to 10, up to 10,000, up to 100,000, up to 1,000,000, etc. third parties(s)). In some embodiments, a third party can be a medical professional, a caretaker/caregiver, or anyone else besides a user that can utilize the data provided by the inventive apparatus to treat the user.

In some embodiments, an embodiment of the apparatus of the present invention is a BGMS configured to have an all-in-one feature which allows for the holding/storage of all relevant items for glucose self-testing in one lancing device enclosure, while enabling blood glucose measurement (e.g., but not limited to, a plurality of lancets, a plurality of test strips, a plurality of control strips, a plurality of control solutions, etc.).

In some embodiments, the present invention is an apparatus which includes a BGMS configured to calculate a quantitative measurement of glucose from a fresh (i.e., lancing within less than 1-5 minutes) capillary whole blood sample(s). In some embodiments, the BGMS is configured to deliver the quantitative measurement of the blood glucose level from at least one whole blood sample drawn from, e.g., but not limited to, a fingertip. In some embodiments, the present invention is an apparatus configured for use by a single user (i.e., patient). In some embodiments, the present invention is a apparatus not for use by more than one user. In some embodiments, the present invention is a system configured for use by plurality of users (e.g., but not limited to, 10,000, 100,000, 1,000,000, 10,000,000, etc.) and third parties (e.g., but not limited to, 10,000, 100,000, 1,000,000, 10,000,000, etc.).

In some embodiments, the present invention is an apparatus which includes a BGMS for self-testing outside a body (i.e., in vitro diagnostic use) by a user diagnosed with diabetes. In some embodiments, the BGMS is configured to be used by a user for monitoring an effectiveness of diabetes control. In some embodiments, the BGMS is not used for the diagnosis of or screening of diabetes or for neonatal use.

In some embodiments, the apparatus of the present invention includes a BGM for use with at least one blood glucose test strip, at least one control solution(s), at least one lancing device, at least one lancet(s), application software, or any combination thereof.

In some embodiments, the present invention is an apparatus which includes a BGM configured to connect to a smart mobile device by use of an audio jack, and further includes smart mobile device application software, a plurality of disposable test strips, a disposable test strip cartridge, control solutions (e.g., but not limited to, M level and H level), a check strip, and a lancing device, a plurality of lancets, or any combination thereof. In some embodiments, the lancing device is configured to include enclosures for housing the BGM and/or the test strip cartridge.

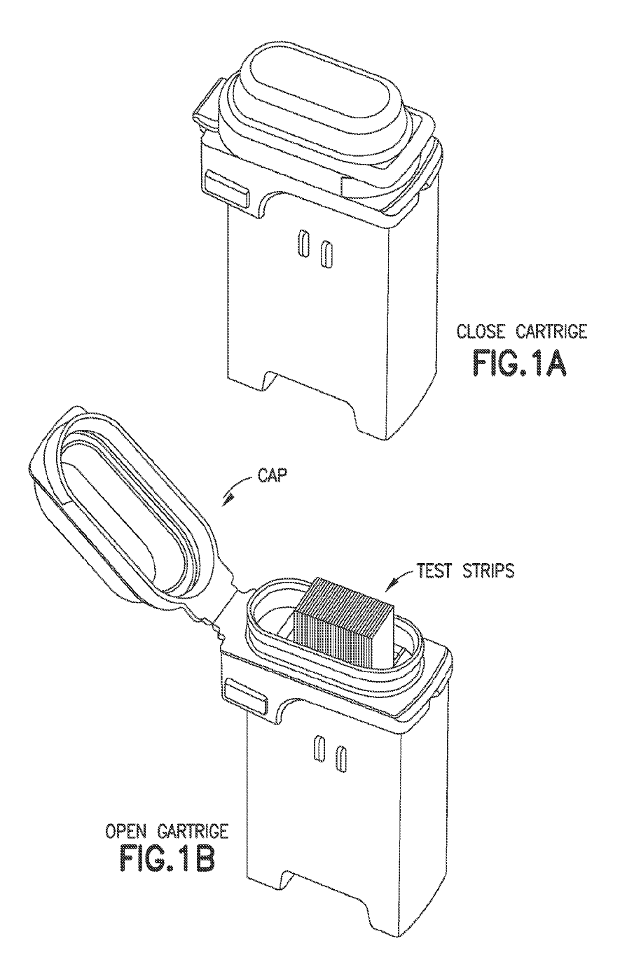

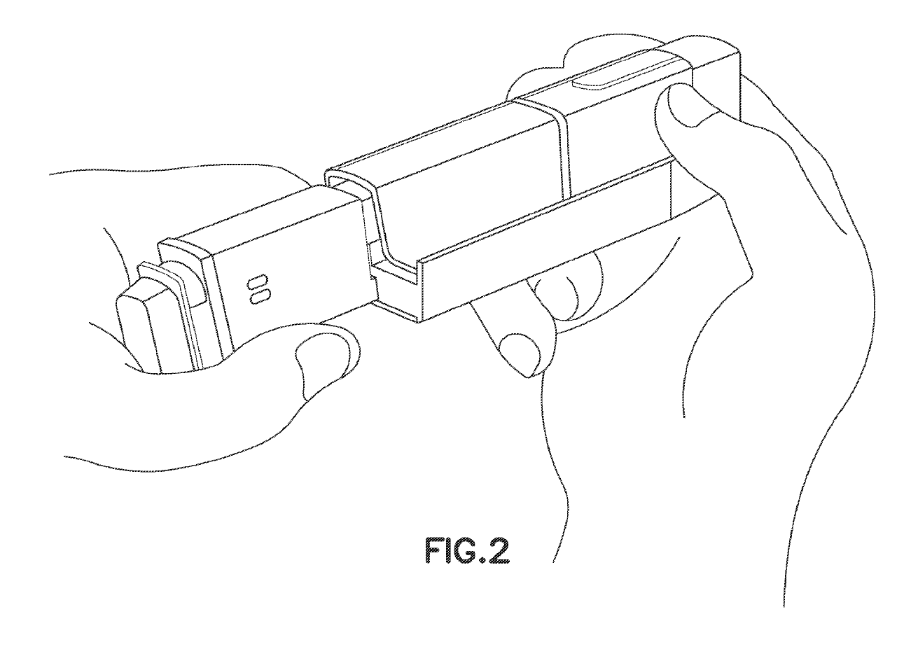

In some embodiments, the present invention further comprises a test strip cartridge comprising a container with an opening cap similar to a vial (FIG. 1) and housed in the lancing device (FIG. 2). In some embodiments, the test strip cartridge is a rounded vial. In some embodiments, the test strip cartridge is an angular vial. In some embodiments, the present invention comprises a cartridge containing, e.g., but not limited to, 25 test strips. In some embodiments, the test strips are housed inside the device in a manner that allows for ejection of one test strip at a time. In some embodiments, the cartridge is housed in the lancing device, and the cartridge is configured for removal and replacement from a lancing device. In some embodiments, the present invention comprises a cartridge cap that can be opened and closed by applying a force comparable to the common test strips rounded vials.

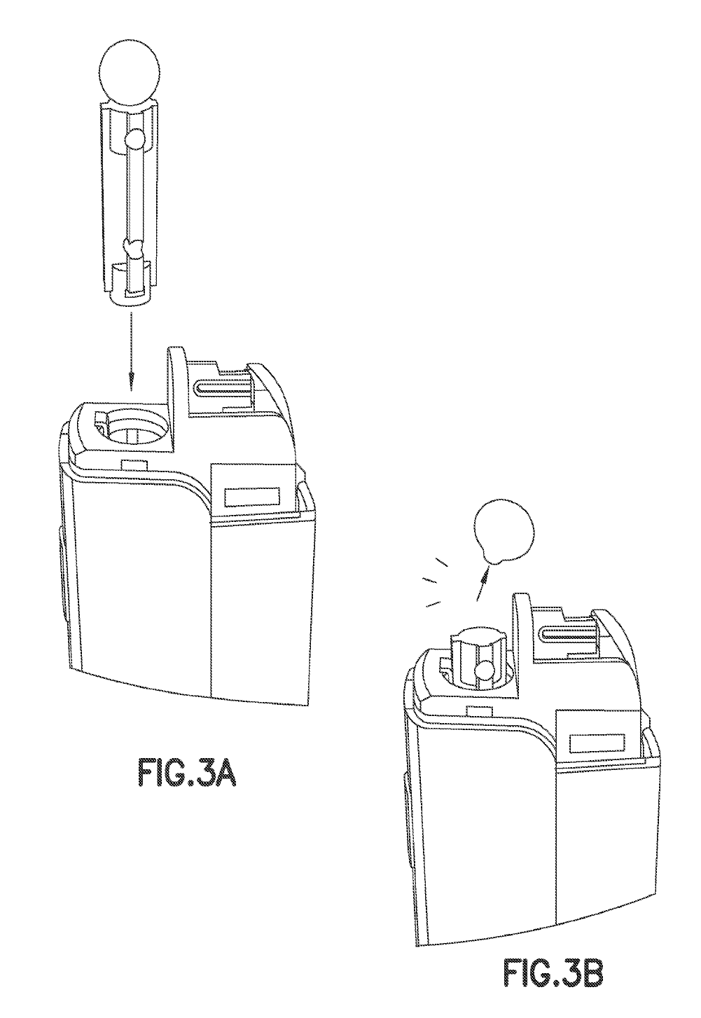

An exemplary embodiment of the apparatus of the present invention is shown in FIG. 3. In some embodiments, the apparatus of the present invention includes a lancing device comprising a lancing mechanism encased in a plastic with a removable cap. In some embodiments, the apparatus of the present invention comprises an enclosure for a BGM (dongle) and a test strip cartridge. In some embodiments, the apparatus of the present invention comprises a plurality of sterile lancets configured to be inserted in the lancing device and replaced by the user. In some embodiments, the apparatus of the present invention comprises sterile lancets.

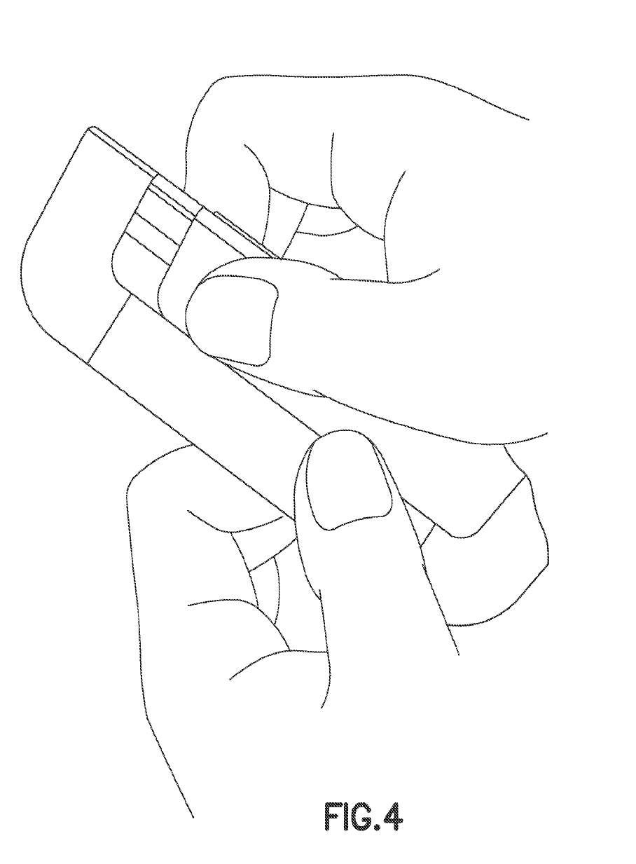

In some embodiments of the apparatus of the present invention, the apparatus includes a lancing device comprising a depth indicator dial that allows adjustment of a lancet depth. In some embodiments, the lancet depth comprises at least four grades of depth. In some embodiments, the lancet depth comprises between 4-10 grades of depth. In some embodiments, the lancet depth comprises between 6-10 grades of depth. In some embodiments, the lancet depth comprises between 8-10 grades of depth. In some embodiments, the lancet depth comprises between 4-8 grades of depth. In some embodiments, the lancet depth comprises between 4-6 grades of depth. An embodiment of the present invention is shown in FIG. 4. In some embodiments of the apparatus of the present invention, a handle is used to cock the lancet.

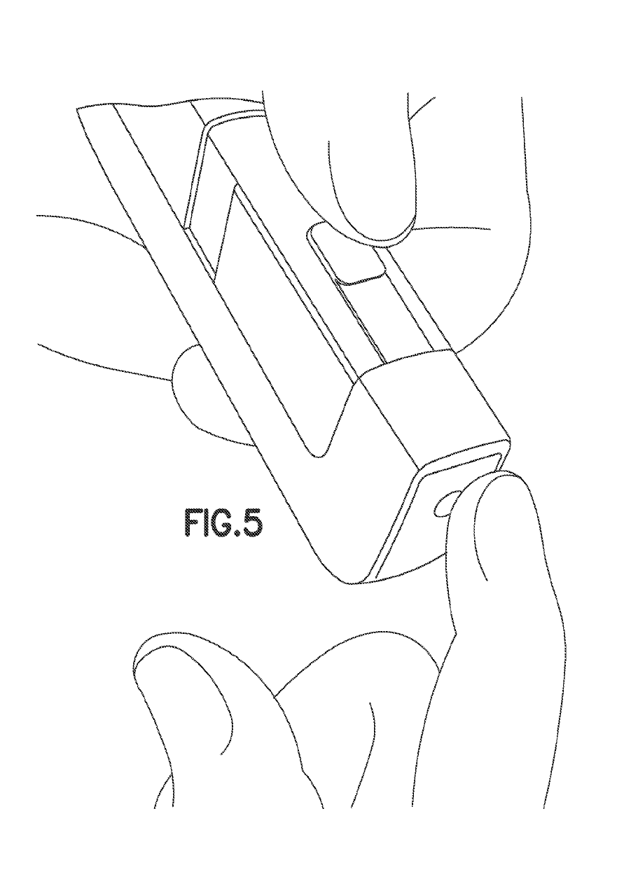

An exemplary embodiment of the apparatus of the present invention is shown in FIG. 5. In some embodiments of the apparatus of the present invention, a button is released to actuate the lancet. In some embodiments of the apparatus of the present invention, a spring automatically returns the lancet into a cover after release.

In some embodiments of the apparatus of the present invention, the lancing device comprises an enclosure for the BGM and the strip cartridge. In some embodiments, each cover is configured for removal and can be re-inserted into the apparatus by the user. In an embodiment, a spring automatically returns a lancet to inside a cover after release of the lancet. In some embodiments, the lancet lever provides a change in needle depth and allows adjustment of needle depth. In another embodiment, the needle depth can be adjusted in four grades. In some embodiments, the lancet penetrates the skin of a user for the purpose of drawing blood. In some embodiments, blood can be drawn at the lowest setting of the lancet lever. In some embodiments, the lancet loading handle is easily cocked by a user. In some embodiments, the lancet release handle is easily actuated by a user. In some embodiments, the lancet replacement handle allows for replacement of the lancet. In some embodiments, the lancing device covers include removal indications. In some embodiments, the dongle remains secure at normal use conditions. In some embodiments, the BGM holder is configured to be open while the BGM is removed from the lancing device.

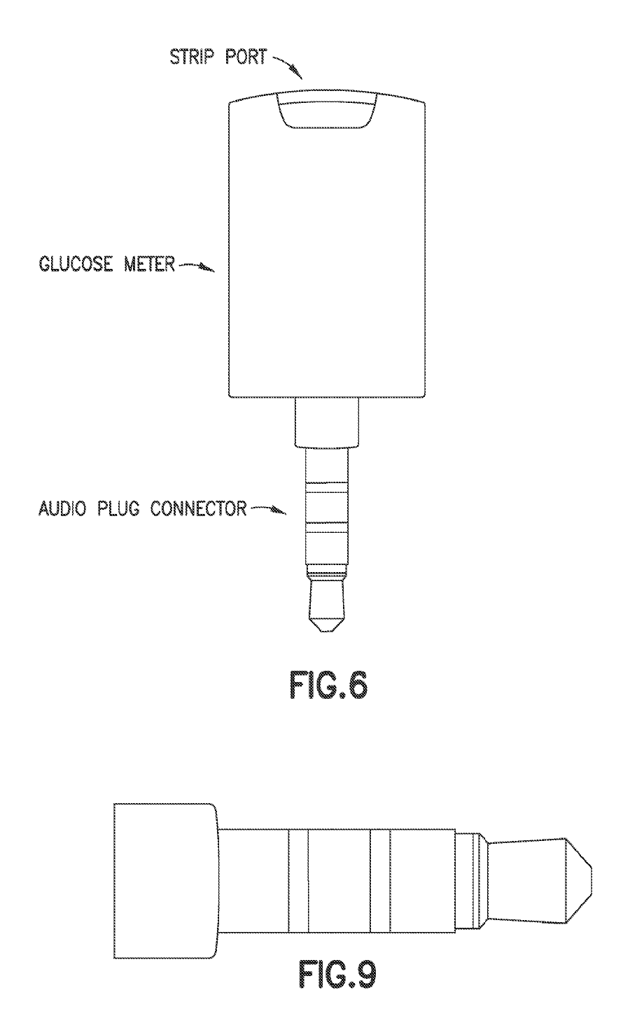

An exemplary embodiment of the apparatus of the invention is shown in FIG. 6. In some embodiments, a BGM (also referred to herein as “dongle”) comprises an electronic component built on top of printed circuit board (PCB) that is configured to read a chemical reaction from a test strip and to communicate with a smart mobile device to present a result of the blood glucose measure to a user.

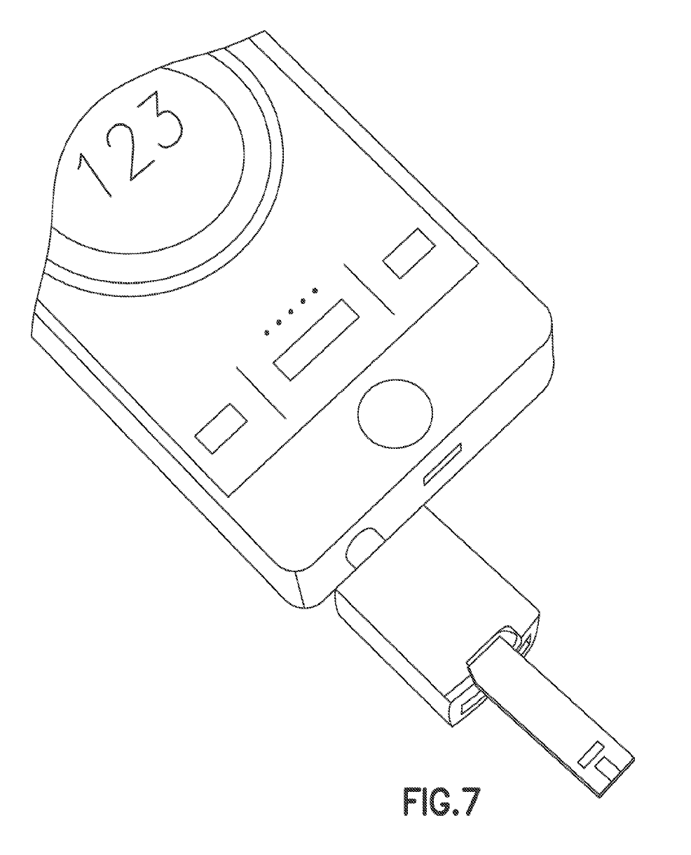

An exemplary embodiment of the apparatus of the present invention is shown in FIG. 7, illustrating the connection between the BGM, a test strip, and a mobile device.

FIG. 8 is an exemplary embodiment of the apparatus of the present invention, shown as a block diagram of the BGM.

An embodiment of the present invention is a BGM comprising a plastic enclosure, a test strip connector, a smart mobile device connector plug, and an electronic PCB that is configured (1) to have a smart mobile device supply power to the BGM electronic, (2) to communicate with the smart mobile device by the audio plug, and (3) to measure the process.

In some embodiments, the apparatus of the present invention includes a BGM configured to use a phone audio jack for bi-directional communication and power generation for the entire circuit. In some embodiments, the audio plug is a 4-pole, 3.5 mm earpiece/microphone connector that utilizes Frequency Shift Keying (FSK) communication signals in the audio frequency range.

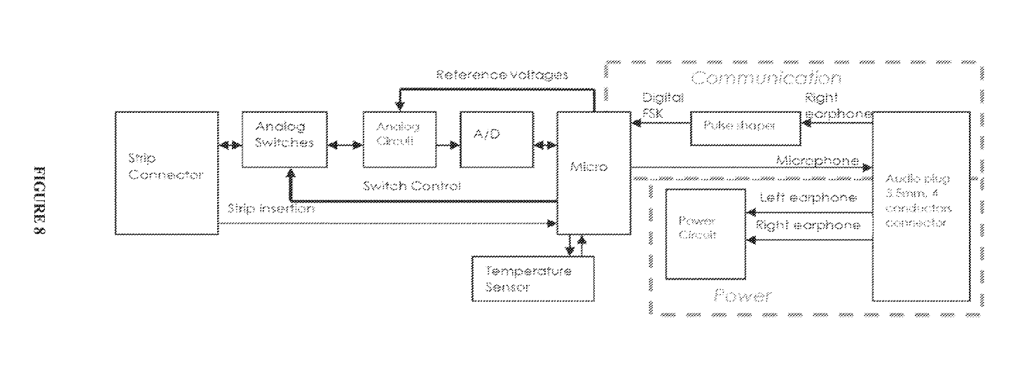

An exemplary embodiment of the apparatus of the present invention is illustrated in FIG. 9. In some embodiments, an audio plug comprises a left audio out from a smart mobile device for power, a right audio out from a smart device for sending data to BGM in FSK and power, a common/ground, and a microphone for getting data from BGM in FSK.

In some embodiments, the apparatus of the present invention is a platform, which can be a diabetes management platform, configured to connect each user of a plurality of users (e.g., but not limited to, 10,000, 100,000, 1,000,000, 10,000,000, 100,000,000), each caregiver of a plurality of caregivers (e.g., but not limited to, 10,000, 100,000, 1,000,000, 10,000,000, 100,000,000), each medical/healthcare professional of a plurality of medical/healthcare professionals (e.g., but not limited to, 10,000, 100,000, 1,000,000, 10,000,000, 100,000,000), or any combination thereof, anywhere in the world.

In some embodiments, the system of the present invention is configured to utilize cloud-based software, allowing a user to record, save, track, analyze, manage, share, or any combination thereof, all or a portion of the user's diabetes-related information in one lifestyle management platform.

In some embodiments, the apparatus of the present invention periodically synchronizes each user's, of a plurality of users, data in to one place, so a user can maintain control of the user's health. In some embodiments, the user's data is synchronized about every second. In some embodiments, the user's data is synchronized about every minute. In some embodiments, the user's data is synchronized about every hour. In some embodiments, the user's data is synchronized about every day. In some embodiments, the user's data is synchronized about every week. In some embodiments, the user's data is synchronized about every month. In some embodiments, the user's data is synchronized about every year. In some embodiments, the synchronization is manual and/or automatic. In some embodiments, the user can initiate synchronization. In an embodiment, the apparatus of the present invention is an all-in-one mobile and cloud based diabetes management platform, with glucose measurement device, data capture and analysis, sharing, and social features designed to fit patients with diabetes everyday life.

In an embodiment, the apparatus of the present invention is configured to provide diabetes monitoring that connects the user, caregiver, healthcare professional, or any combination thereof, anywhere in the world.

FIGS. 1A and 1B illustrate an embodiment of the test strip cartridge of the present invention.

FIG. 2 illustrates an embodiment of the test strip cartridge housed in the lancing device of the present invention.

FIGS. 3A and 3B illustrate an embodiment the apparatus of the present invention, where the lancet being inserted into a lancing device of the present invention.

FIG. 4 illustrates an embodiment of the present invention, where the lancet is placed in the lancing device.

FIG. 5 illustrates an embodiment of the present invention, where the Lancet is released into the lancing device.

FIG. 6 illustrates an embodiment of the BGM (Dongle) of the present invention.

FIG. 7 illustrates an embodiment of the BGM, test strip, and smart mobile device of the present invention.

FIG. 8 illustrates a block diagram showing an embodiment of the BGM (dongle) of the present invention.

FIG. 9 illustrates an embodiment of a glucose meter audio plug of the present invention.

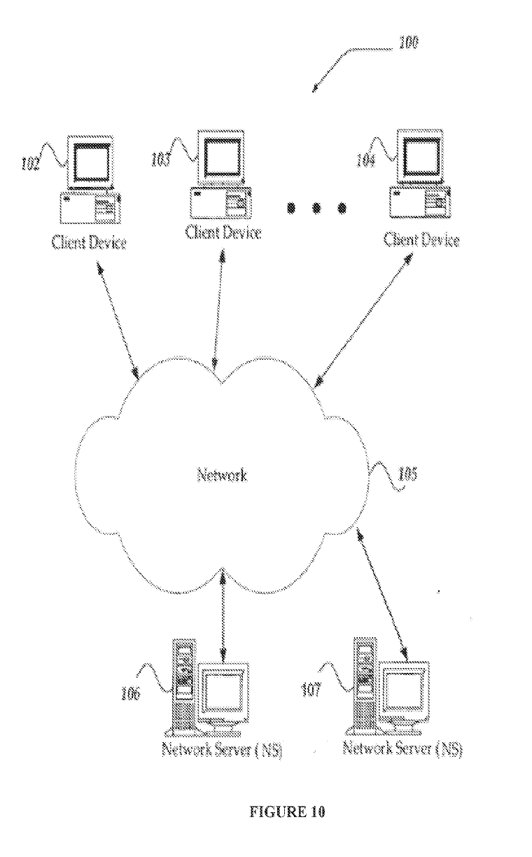

FIGS. 10 and 11 illustrates some embodiments of the inventive system of the present invention, showing users with type 1 diabetes connecting over network servers in accordance with the invention.

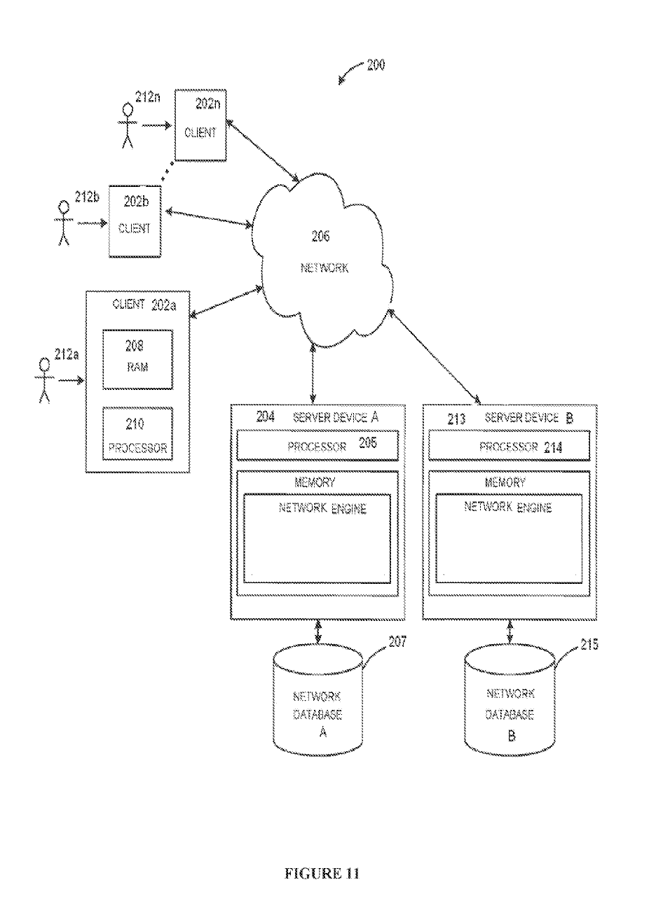

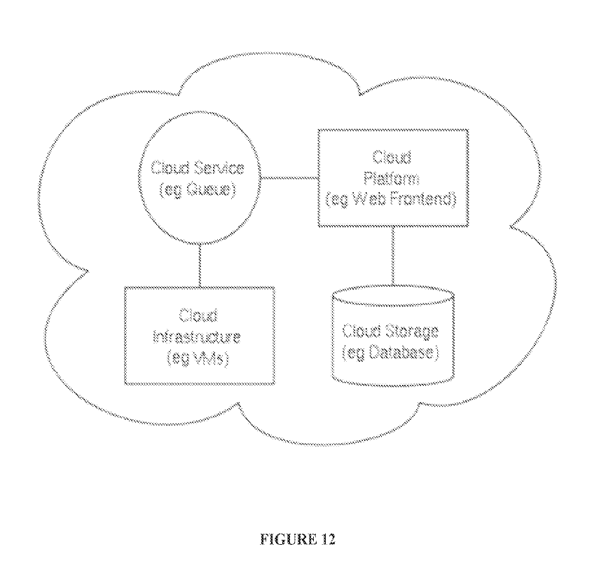

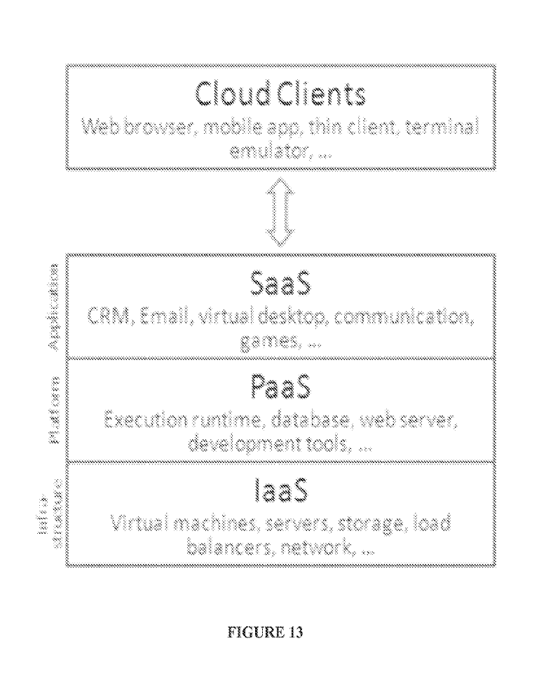

FIGS. 12-13 illustrate further aspects of some embodiments of the system of the present invention.

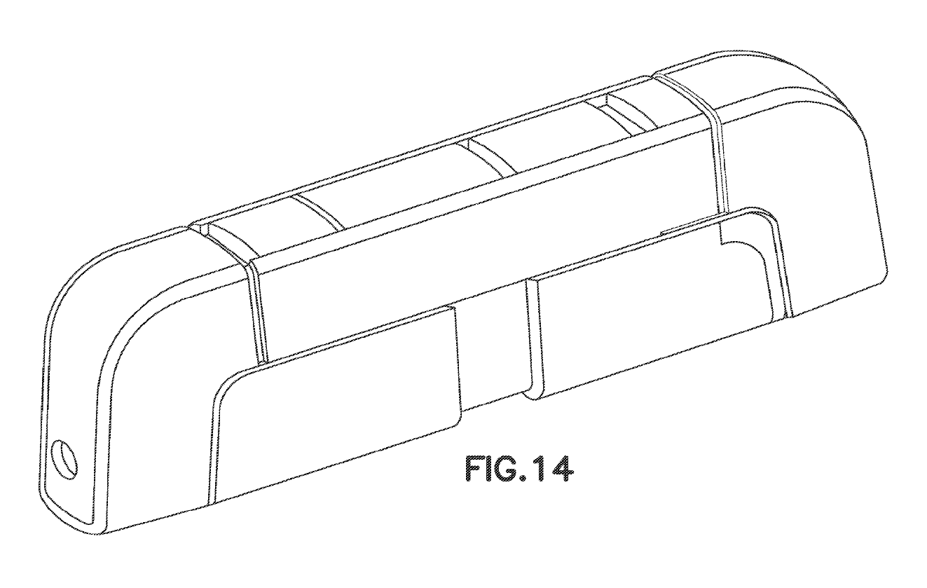

FIG. 14 illustrates an embodiment of the apparatus of the present invention, where the apparatus is in a closed position.

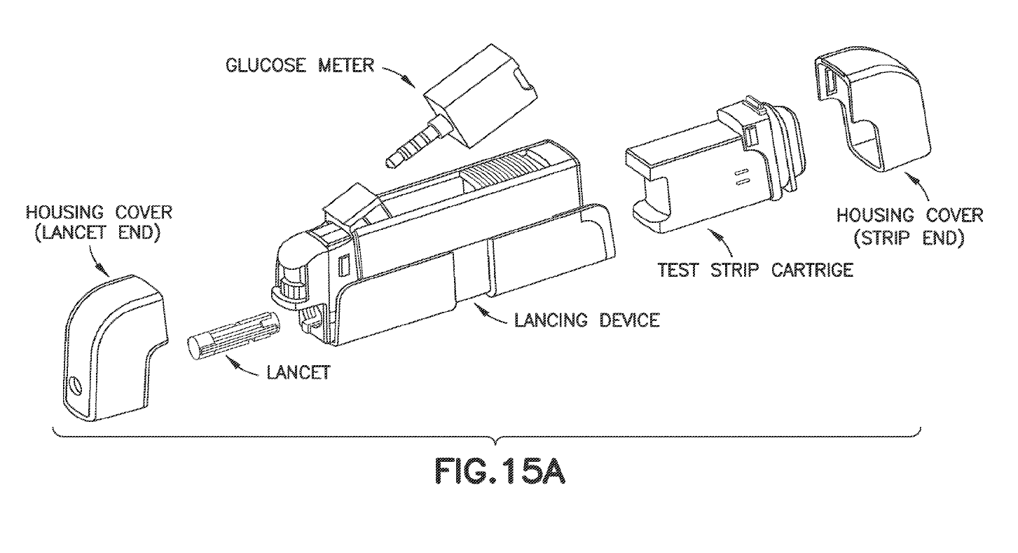

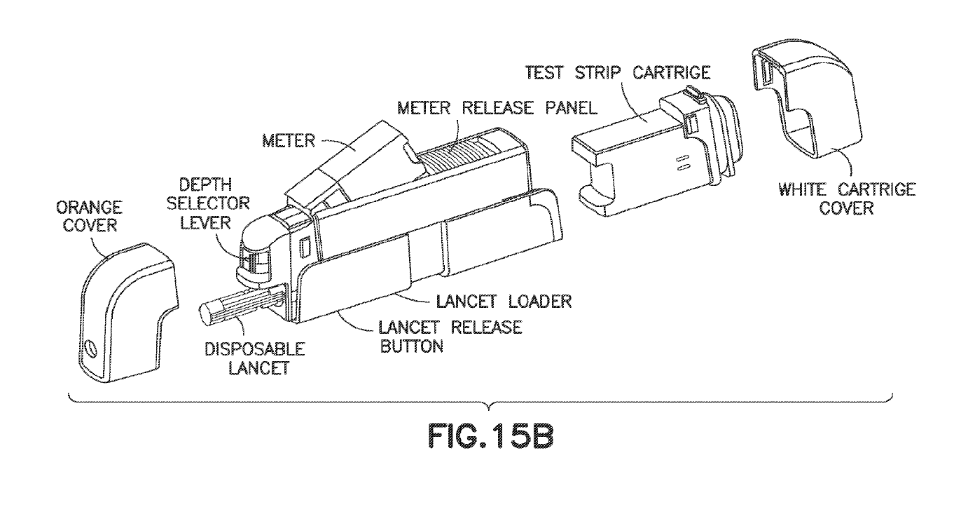

FIGS. 15A and 15B are exploded views of embodiments of the device components of the apparatus of the present invention. FIG. 15B shows an exemplary embodiment of the glucose meter (FIG. 15B, the meter (A) and the meter release panel (B)), where the glucose meter is an audio connector that can be plugged/attached into a headphone socket of the smart mobile device and a strip port for insertion of the test strip, where the glucose meter receives power from the mobile device. In an embodiment, the glucose meter does not require batteries or any other electrical source. In an embodiment, when not in use, the meter snaps into its slot in the housing for protected storage.

FIG. 15B also shows embodiments of the apparatus of the present invention, including the lancing device and cover (FIG. 15B, the cover (C), disposable lancet (D), lancet release button (E), lancet loader (F), and depth selector lever (G)), where the lancing device is built into the housing and uses disposable lancets. In some embodiments, the lancing device cover prevents the user from unintentional punctures. In some embodiments, lancets are changed for each use. In some embodiments, puncture depth may be adjusted by removing the cover and moving the depth selection dial, and the cover can then be replaced for use.

FIG. 15B further shows an exemplary embodiment of the apparatus of the present invention, showing a test strip cartridge (FIG. 15B, test strip cartridge (H)), where the test strip cartridge can hold 25 test strips, and the cartridge can snap into the housing of the apparatus.

FIG. 15B further shows an exemplary embodiment of the apparatus of the present invention, showing the use of a 30G lancet. In some embodiments, the lancet is replaced by removing the lancing device cover, and pulling out the used lancet and replacing the used lance with a sterile lancet.

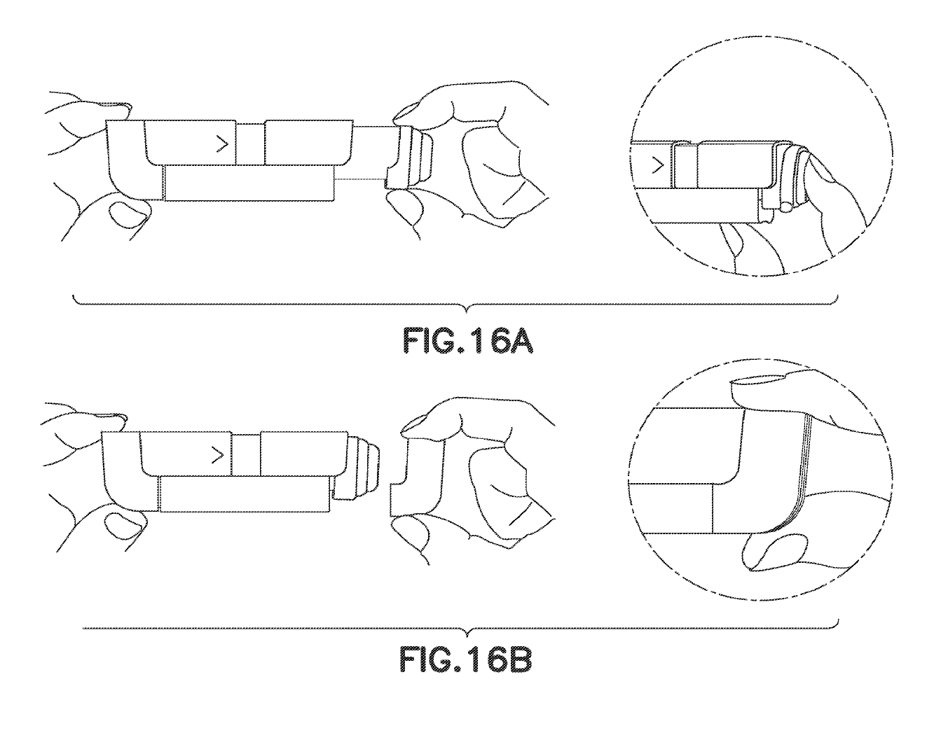

FIGS. 16A and 16B show exemplary embodiments of the apparatus of the present invention, showing insertion of the test strip cartridge. To insert the test strip cartridge, (i) the carbon paper is removed from the device and the cover (i.e., white cover of FIG. 15B) is opened; (ii) the new test strip cartridge is removed from the pouch; (iii) the test strip cartridge is held with the side that opens, facing up (see, e.g., FIG. 16A); (iv) the cartridge is inserted into the housing until it snaps into place; (v) the housing cover is replaced by sliding it into place until it click (see, e.g., FIG. 16B). In an embodiment, if the cover does not close properly, the cartridge may not have been inserted correctly.

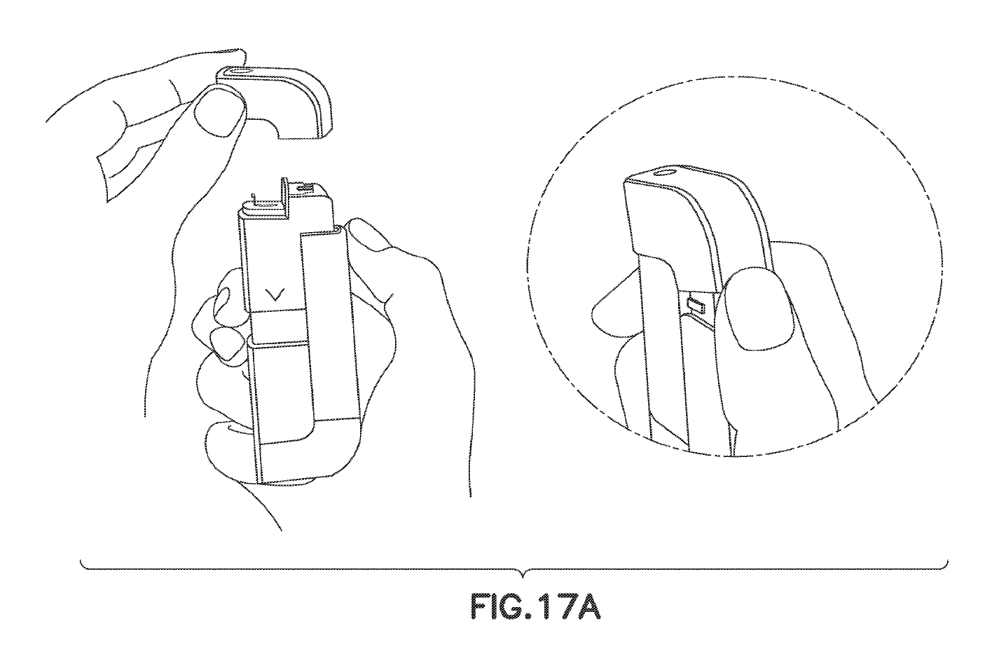

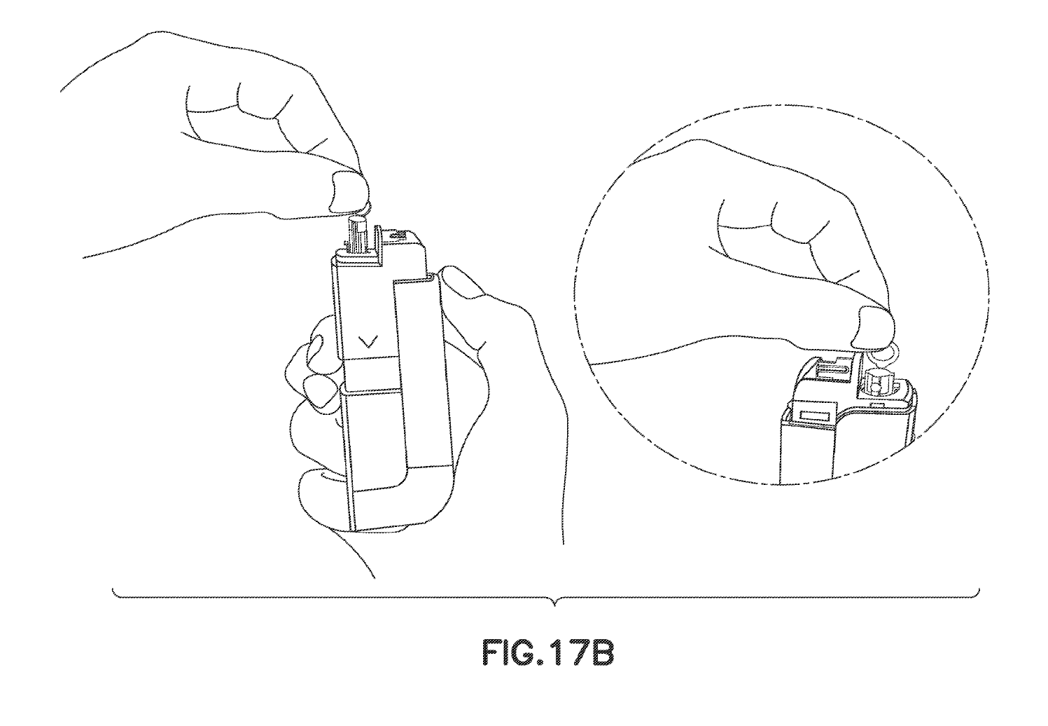

FIGS. 17A and 17B show exemplary embodiments of the apparatus of the present invention, showing insertion of a lancet. To insert the lancet, (i) the lancet device cover is removed (see, e.g., orange cover, FIG. 15B) from the housing, (ii) a new/unused lancet is removed from a packaging, (iii), a sterile lancet is inserted as far as it will go and feels firmly set to a user, (iv) the lancet cap is twisted off without bending the lancet. In some embodiments, the depth of the lancet, as it punctures the skin, can be adjusted by moving the lever between the numbers one to four (printed on the dial), where (1) is the shallowest depth and (4) is the deepest.

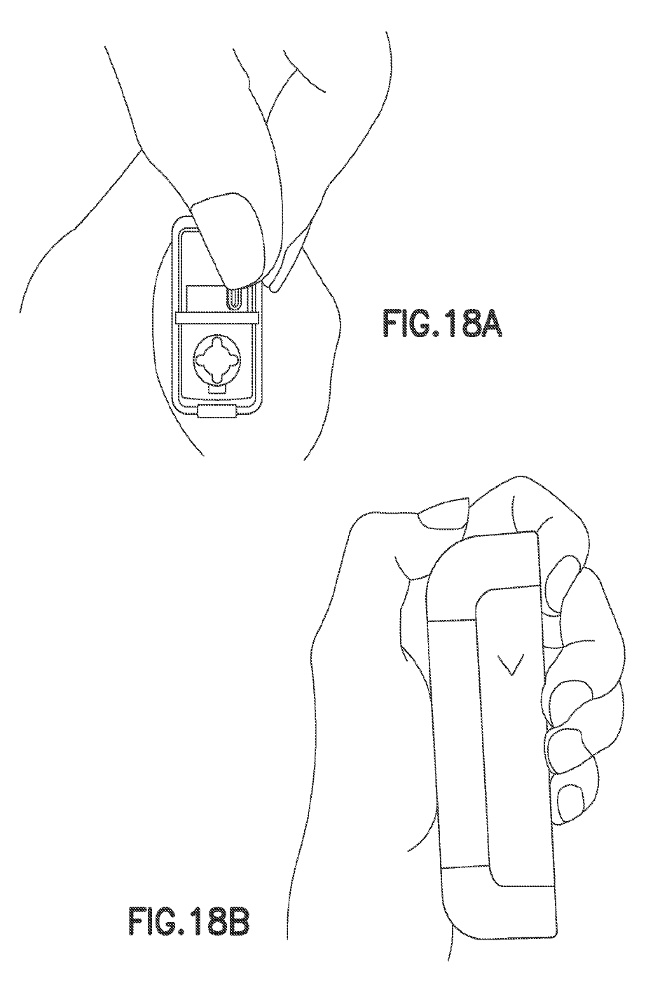

FIGS. 18A and 18B show exemplary embodiments of the apparatus of the present invention, showing setting the lancet depth, where FIG. 18A shows the lancing device lever is moved such that the line on the lever is aligned with the correct depth, and FIG. 18B the lancing device cover is returned by sliding it into place until it clicks, where the user does not touch or bend the lancet.

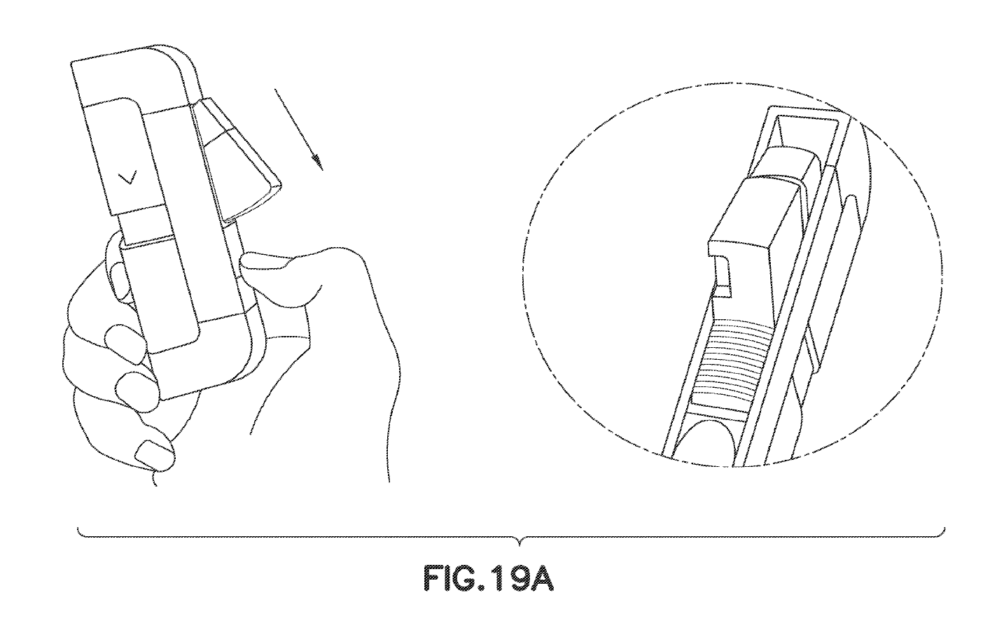

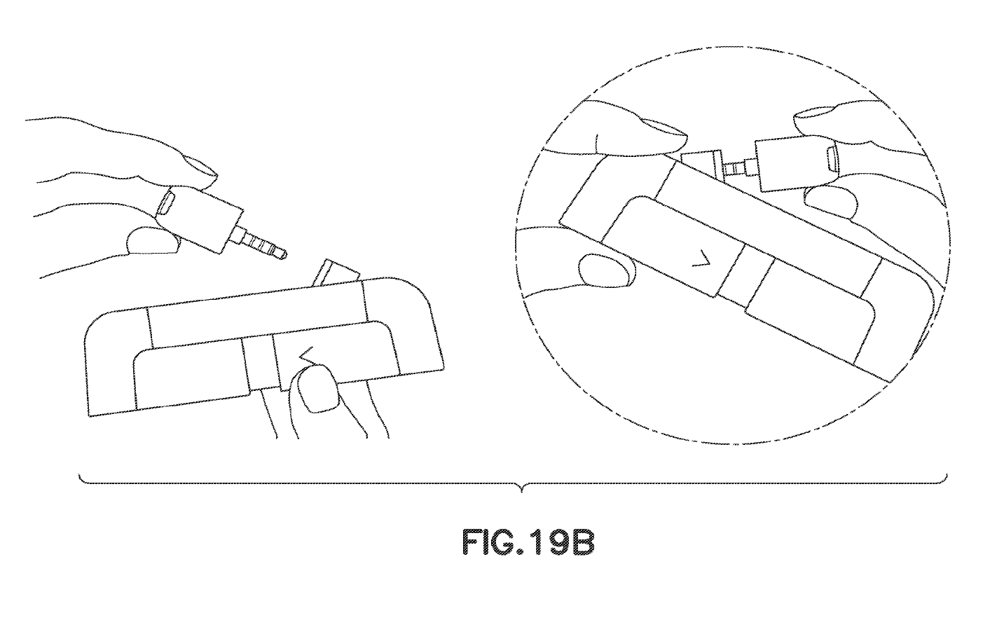

FIGS. 19A and 19B show exemplary embodiments of the apparatus of the present invention, showing how a user can perform a control solution test. In some embodiments, the glucose meter is stored in the housing when not in use. FIG. 19A shows a user removing the meter from the housing by (i) holding the housing so that the ridged panel (e.g., the orange panel of FIG. 15B) is close to user and the end with the lancing device is facing away from the user, and (ii) sliding the user's thumb back ont eh ridged panel, allowing a meter to pop up. FIG. 19B shows a user grasping the meter with two figures and removing the meter from housing.

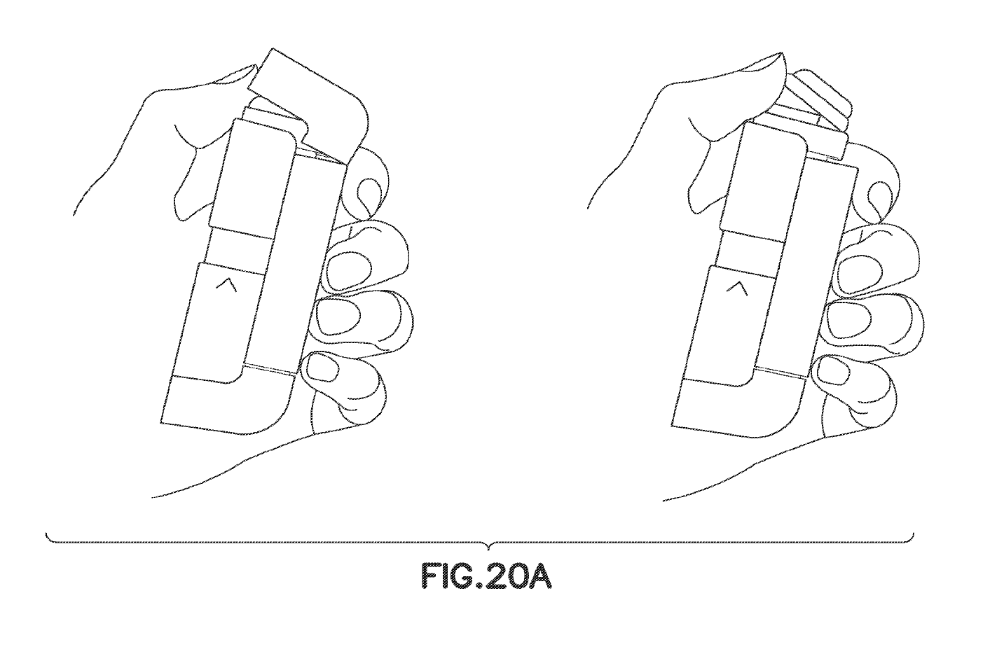

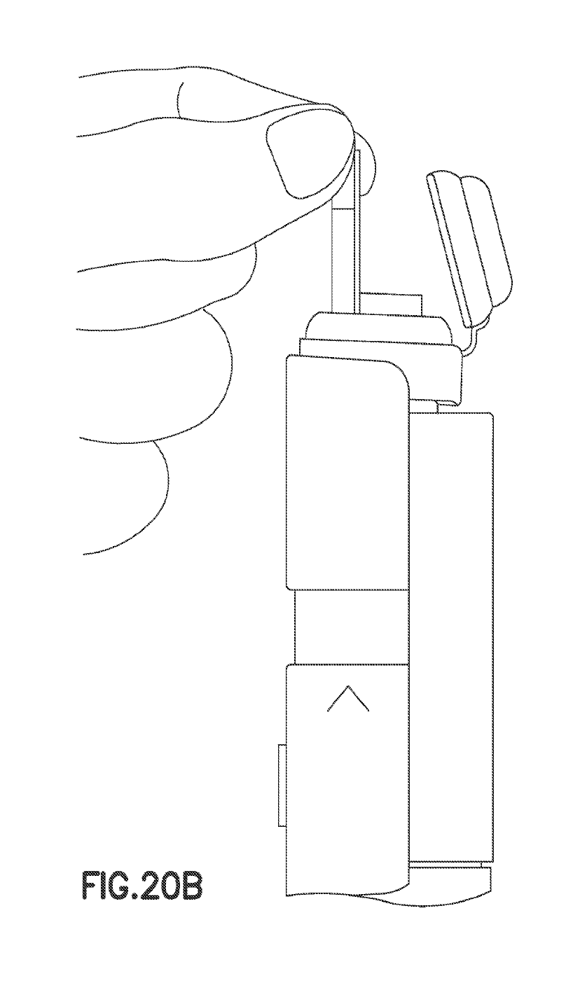

FIGS. 20A and 20B show exemplary embodiments of the apparatus of the present invention, showing the removal of a test strip from the cartridge. FIG. 20A shows that a user first removes the cartridge cover housing by pushing it outwards until it comes off. In an embodiment, the case is opened by pushing up the edge. FIG. 20B shows that the test strip can be removed from the cartridge. In some embodiments, the case can be snap closed (immediately after use) and the cartridge cover can be replaced by pushing it into place until it produces a click.

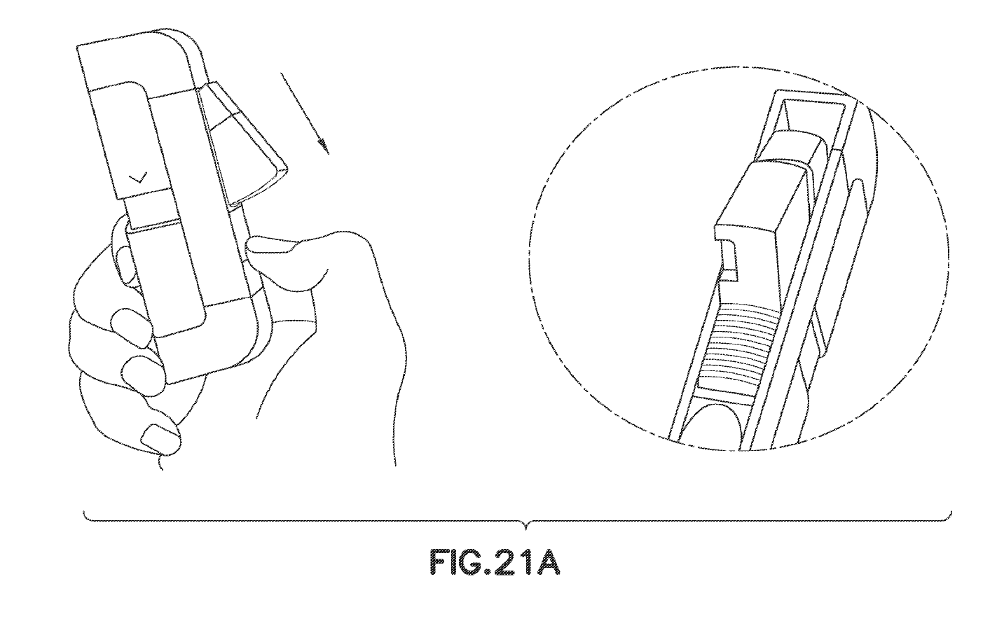

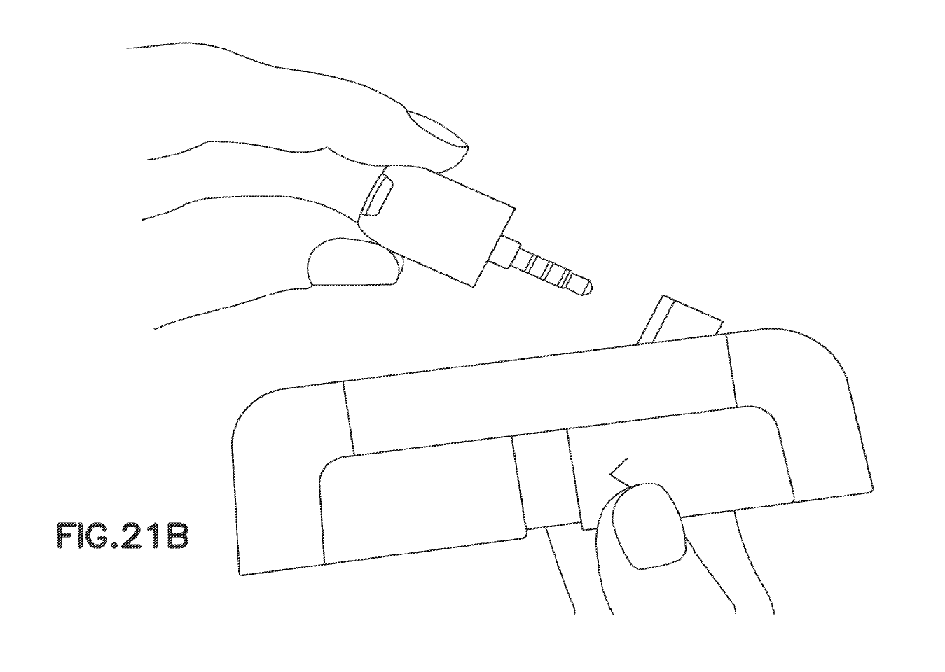

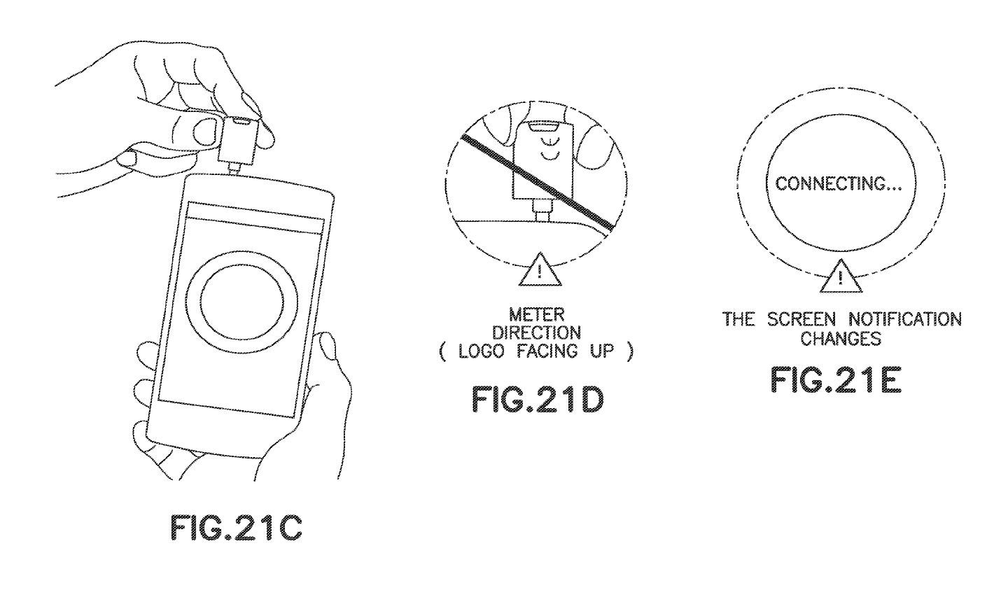

FIGS. 21A-21E show exemplary embodiments of the apparatus of the present invention, showing the method of removing the glucose meter from the housing. FIG. 21A shows the user (i) holding the housing so that the ridged orange panel is close to the user and the end with the lancing device is facing away from the user and (ii) sliding the user's thumb back on the ridged orange panel in the arrow direction, allowing for the meter to pop up. FIG. 21B shows the user grasping the meter with two fingers while removing the meter from the housing, so that the meter can, e.g., be plugged into a smart mobile device audio socket. In some embodiments, the user can tap an application icon on the user's smart mobile device to launch the application prior to connecting with the glucose meter. FIG. 21C shows the user inserting the glucose meter into the audio plug of the smart mobile device in the correct direction. FIG. 21D shows an incorrect direction for connecting the meter to the smart device. FIG. 21E shows a screen notification, indicating that the application is active.

In some embodiments, the glucose meter only requires at least 0.3 uL of blood to perform the test. In some embodiments, the blood is applied to fill the window so that the window turns completely red. In some embodiments, if too little blood is applied to the test strip, an alert informs the user that the sample size is too small and the user should try again with a new strip. In some embodiments, the blood glucose results are automatically saved to memory, and may remain on the screen for a period of time (e.g., but not limited to 30 seconds, 1 minute, 5 minutes, 10 minutes, 15 minutes, 20 minutes, 30 minutes, 45 minutes, 60 minutes, etc.)

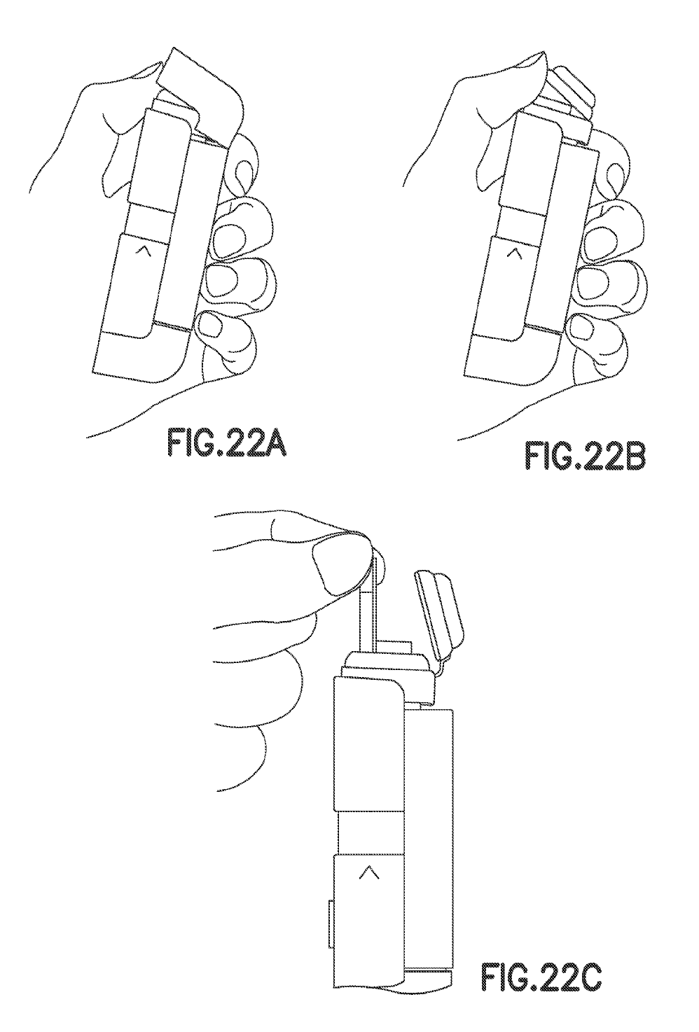

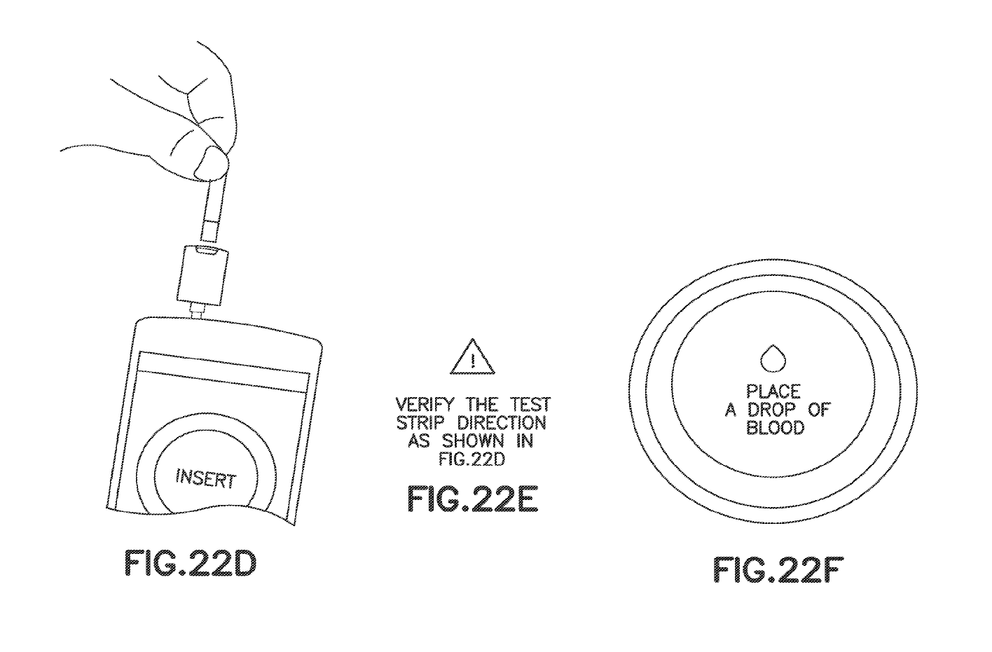

FIGS. 22A-22F show exemplary embodiments of the apparatus of the present invention, showing a method for removing a test strip from the cartridge. FIG. 22A shows the method for removing the cartridge cover of the apparatus housing by pushing it outwards until it comes off (e.g., but not limited to, using the removal indication lines). FIG. 22B shows the method for opening the lid by pushing up the edge. FIG. 22C shows the user removing a test strip from the cartridge. The lid and cartridge cover are closed, and the user holds the test strip with the gold contacts facing upward. FIG. 22D shows the user, without bending the test strip, inserting the test strip into the test strip port of the glucose meter. FIG. 22E shows a prompt delivered by the application to the user. FIG. 22F shows an instruction delivered by the application to a user.

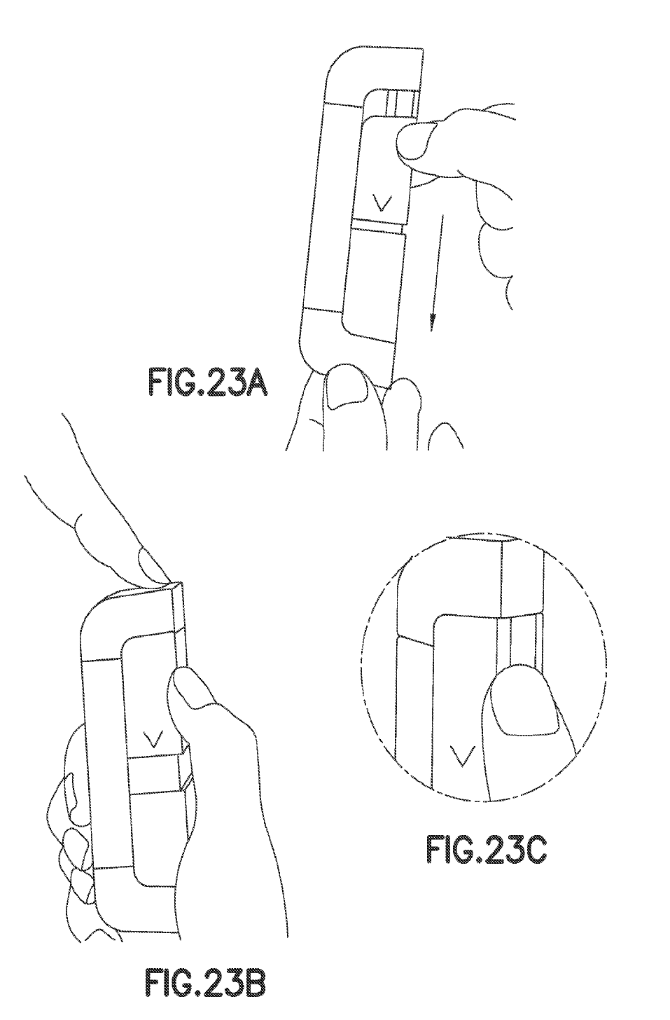

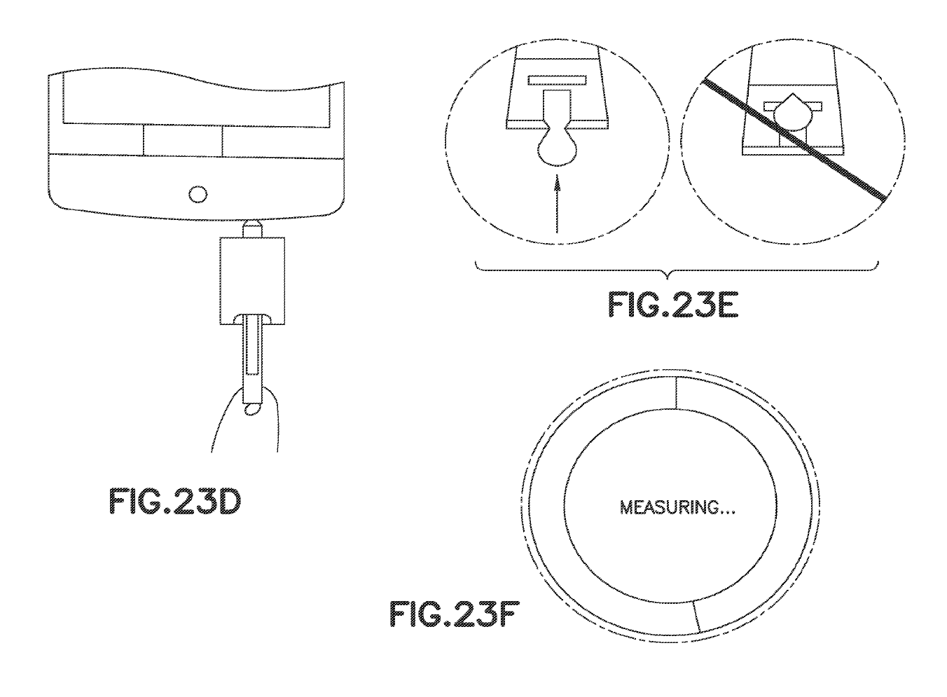

FIGS. 23A-23F show exemplary embodiments of the apparatus of the present invention, showing the user performing the glucose test. FIG. 23A shows the user setting the lancing device for activation by sliding it with, e.g., two fingers. FIG. 23B shows the user placing the user's fingertip on the lancing device cover, and FIG. 23C shows the user pressing the release button to prick the user's finger. FIGS. 23D and 23E show the user applying a drop of blood to the tip of the window of the test strip, allowing capillary action to pull the blood into the test strip. FIG. 23F shows a message delivered to a user when the meter detects that blood has been applied, indicating that the test is in progress. In some embodiments, the user removes the user's finger from the test strip once the message appears. In some embodiments, the glucose test result appears on the screen of the smart device after about between 1-10 seconds (e.g., 6 seconds). In some embodiments, the glucose test result is automatically saved to memory. In some embodiments, a user can remove the test strip and disposable cover once the test is complete, remove the lancet from the lancing device and dispose the used lancet, return the meter to the housing, or any combination thereof.

### Illustrative Operating Environments

FIG. 10 illustrates one embodiment of an environment in which the present invention may operate. However, not all of these components may be required to practice the invention, and variations in the arrangement and type of the components may be made without departing from the spirit or scope of the present invention. In some embodiments, the system and method may include a large number of members and/or concurrent transactions. In other embodiments, the system and method are based on a scalable computer and network architecture that incorporates varies strategies for assessing the data, caching, searching, and database connection pooling. An example of the scalable architecture is an architecture that is capable of operating multiple servers.

In embodiments, members of the computer system 102-104 include virtually any computing device capable of receiving and sending a message over a network, such as network 105, to and from another computing device, such as servers 106 and 107, each other, and the like. In embodiments, the set of such devices includes devices that typically connect using a wired communications medium such as personal computers, multiprocessor systems, microprocessor-based or programmable consumer electronics, network PCs, and the like. In embodiments, the set of such devices also includes devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile device, and the like. Similarly, in embodiments, client devices 102-104 are any device that is capable of connecting using a wired or wireless communication medium such as a PDA, POCKET PC, wearable computer, and any other device that is equipped to communicate over a wired and/or wireless communication medium.

In embodiments, each member device within member devices 102-104 may include a browser application that is configured to receive and to send web pages, and the like. In embodiments, the browser application may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In embodiments, programming may include either Java, .Net, QT, C, C++ or other suitable programming language.

In embodiments, member devices 102-104 may be further configured to receive a message from another computing device employing another mechanism, including, but not limited to email, Short Message Service (SMS), Multimedia Message Service (MMS), instant messaging (IM), internet relay chat (IRC), mlRC, Jabber, push notifications, and the like or a Proprietary protocol.

In embodiments, network 105 may be configured to couple one computing device to another computing device to enable them to communicate. In some embodiments, network 105 may be enabled to employ any form of computer readable media for communicating information from one electronic device to another. Also, in embodiments, network 105 may include a wireless interface, and/or a wired interface, such as the Internet, in addition to local area networks (LANs), wide area networks (WANs), direct connections, such as through a universal serial bus (USB) port, other forms of computer-readable media, or any combination thereof. In embodiments, on an interconnected set of LANs, including those based on differing architectures and protocols, a router may act as a link between LANs, enabling messages to be sent from one to another.

Also, in some embodiments, communication links within LANs typically include twisted wire pair or coaxial cable, while communication links between networks may utilize analog telephone lines, full or fractional dedicated digital lines including T1, T2, T3, and T4, Integrated Services Digital Networks (ISDNs), Digital Subscriber Lines (DSLs), wireless links including satellite links, or other communications links known to those skilled in the art. Furthermore, in some embodiments, remote computers and other related electronic devices could be remotely connected to either LANs or WANs via a modem and temporary telephone link. In essence, in some embodiments, network 105 includes any communication method by which information may travel between client devices 102-104, and servers 106 and 107.

FIG. 11 shows another exemplary embodiment of the computer and network architecture that supports the method and system. The member devices 202a, 202b thru 202n shown each at least includes a computer-readable medium, such as a random access memory (RAM) 208 coupled to a processor 210 or FLASH memory. The processor 210 may execute computer-executable program instructions stored in memory 208. Such processors comprise a microprocessor, an ASIC, and state machines. Such processors comprise, or may be in communication with, media, for example computer-readable media, which stores instructions that, when executed by the processor, cause the processor to perform the steps described herein. Embodiments of computer-readable media may include, but are not limited to, an electronic, optical, magnetic, or other storage or transmission device capable of providing a processor, such as the processor 210 of client 202a, with computer-readable instructions. Other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. The instructions may comprise code from any computer-programming language, including, for example, C, C++, C#, Visual Basic, Java, Python, Perl, and JavaScript

Member devices 202a-n may also comprise a number of external or internal devices such as a mouse, a CD-ROM, DVD, a keyboard, a display, or other input or output devices. Examples of client devices 202a-n may be personal computers, digital assistants, personal digital assistants, cellular phones, mobile phones, smart phones, pagers, digital tablets, laptop computers, Internet appliances, and other processor-based devices. In general, a client device 202a may be any type of processor-based platform that is connected to a network 206 and that interacts with one or more application programs. Client devices 202a-n may operate on any operating system capable of supporting a browser or browser-enabled application, such as Microsoft™, Windows™, or Linux. The client devices 202a-n shown may include, for example, personal computers executing a browser application program such as Microsoft Corporation's Internet Explorer™, Apple Computer, Inc.'s Safari™, Mozilla Firefox, and Opera. Through the client devices 202a-n, users, 212a-n communicate over the network 206 with each other and with other systems and devices coupled to the network 206. As shown in FIG. 11B, server devices 204 and 213 may be also coupled to the network 206.

In some embodiments, the term “mobile electronic device” may refer to any portable electronic device that may or may not be enabled with location tracking functionality. For example, a mobile electronic device can include, but is not limited to, a mobile phone, Personal Digital Assistant (PDA), Blackberry™, Pager. Smartphone, or any other reasonable mobile electronic device. For ease, at times the above variations are not listed or are only partially listed, this is in no way meant to be a limitation.

In some embodiments, the terms “proximity detection,” “locating,” “location data,” “location information,” and “location tracking” as used herein may refer to any form of location tracking technology or locating method that can be used to provide a location of a mobile electronic device, such as, but not limited to, at least one of location information manually input by a user, such as, but not limited to entering the city, town, municipality, zip code, area code, cross streets, or by any other reasonable entry to determine a geographical area; Global Positions Systems (GPS); GPS accessed using Bluetooth™; GPS accessed using any reasonable form of wireless and/or non-wireless communication; WiFi™ server location data; Bluetooth™ based location data; triangulation such as, but not limited to, network based triangulation, WiFi™ server information based triangulation, Bluetooth™ server information based triangulation; Cell Identification based triangulation, Enhanced Cell Identification based triangulation, Uplink-Time difference of arrival (U-TDOA) based triangulation, Time of arrival (TOA) based triangulation, Angle of arrival (AOA) based triangulation; techniques and systems using a geographic coordinate system such as, but not limited to, longitudinal and latitudinal based, geodesic height based, cartesian coordinates based; Radio Frequency Identification such as, but not limited to, Long range RFID, Short range RFID; using any form of RFID tag such as, but not limited to active RFID tags, passive RFID tags, battery assisted passive RFID tags; or any other reasonable way to determine location. For ease, at times the above variations are not listed or are only partially listed, this is in no way meant to be a limitation.

In some embodiments, near-field wireless communication (NFC) can represent a short-range wireless communications technology in which NFC-enabled devices are “swiped,” “bumped,” “tap” or otherwise moved in close proximity to communicate. In some embodiments, NFC could include a set of short-range wireless technologies, typically requiring a distance of 10 cm or less.

In some embodiments, NFC may operate at 13.56 MHz on ISO/IEC 18000-3 air interface and at rates ranging from 106 kbit/s to 424 kbit/s. In some embodiments, NFC can involve an initiator and a target; the initiator actively generates an RF field that can power a passive target. In some embodiment, this can enable NFC targets to take very simple form factors such as tags, stickers, key fobs, or cards that do not require batteries. In some embodiments, NFC peer-to-peer communication can be conducted when a plurality of NFC-enable devices within close proximity of each other.

For purposes of the instant description, the terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user). In some embodiments, the instant invention offers/manages the cloud computing/architecture as, but not limiting to: infrastructure a service (IaaS), platform as a service (PaaS), and software as a service (SaaS). FIGS. 12 and 13 illustrate schematics of exemplary implementations of the cloud computing/architecture.

Of note, the embodiments described herein may, of course, be implemented using any appropriate computer system hardware and/or computer system software. In this regard, those of ordinary skill in the art are well versed in the type of computer hardware that may be used (e.g., a mainframe, a mini-computer, a personal computer (“PC”), a network (e.g., an intranet and/or the internet)), the type of computer programming techniques that may be used (e.g., object oriented programming), and the type of computer programming languages that may be used (e.g., C++, Basic, AJAX, Javascript). The aforementioned examples are, of course, illustrative and not restrictive.

In some embodiments, the instant invention provides for an apparatus, including: a lancing device; a cartridge; where the lancing device is configured to house the cartridge; where the cartridge is configured to house a plurality of test strips and a glucose monitoring apparatus; where the glucose monitoring apparatus includes: a connecting plug configured to mate with an audio jack phone port, where the audio jack phone port has at least three audio pins; a first ring, where the first ring is positioned on the connecting plug, and where the first ring is configured to mate with a first audio pin of the audio jack phone port; a second ring, where the second ring is positioned on the connecting plug, and where the second ring is configured to mate with a second audio pin of the audio jack phone port; and a third ring, where the third ring is positioned on the connecting plug, and where the third ring is configured to mate with a third audio pin of the audio jack phone port; where at least one of the first, second, and third rings of the connecting plug is configured to receive power for the glucose monitoring apparatus from the audio jack phone port, where at least one of the first, second, and third rings of the connecting plug is configured to receive data from the audio jack phone port, where at least one of the first, second, and third rings of the connecting is configured to transmit data from the glucose monitoring apparatus to the audio jack phone port; and where the glucose monitoring apparatus is configured to determine a glucose test result from at least one test strip of the plurality of test strips. In some embodiments, the apparatus is configured to house at least one test strip. In some embodiments, the apparatus further houses a plurality of test strips. In some embodiments, the apparatus is configured to deploy a lancet of the plurality of lancets and automatically return the lancet of the plurality of lancets into the housing. In some embodiments, the apparatus further includes a button configured to release the lancet. In some embodiments, the apparatus further includes a test strip connector configured to read the test strip glucose level. In some embodiments, the lancing device is configured to eject a lancet. In some embodiments, the apparatus further includes a depth indicator dial configured to allow for adjustment of a lancet penetration depth. In some embodiments, the apparatus further includes a first housing cover configured to cover the disposable lancet. In some embodiments, the apparatus further includes a second housing cover configured to attach to the lancing device at a test strip end position.

In some embodiments, the instant invention provides for a computer system, including: at least one server having software stored on a non-transient computer readable medium; where, upon execution of the software, the at least one server is at least configured to receive glucose test data from a plurality of apparatuses, where each apparatus includes: a lancing device; a cartridge; where the lancing device is configured to house the cartridge; where the cartridge is configured to house a plurality of test strips and a glucose monitoring apparatus; where the glucose monitoring apparatus includes: a connecting plug configured to mate with an audio jack phone port, where the audio jack phone port has at least three audio pins; a first ring, where the first ring is positioned on the connecting plug, and where the first ring is configured to mate with a first audio pin of the audio jack phone port; a second ring, where the second ring is positioned on the connecting plug, and where the second ring is configured to mate with a second audio pin of the audio jack phone port; and a third ring, where the third ring is positioned on the connecting plug, and where the third ring is configured to mate with a third audio pin of the audio jack phone port; where at least one of the first, second, and third rings of the connecting plug is configured to receive power for the glucose monitoring apparatus from the audio jack phone port, where at least one of the first, second, and third rings of the connecting plug is configured to receive data from the audio jack phone port, where at least one of the first, second, and third rings of the connecting is configured to transmit data from the glucose monitoring apparatus to the audio jack phone port; and where the glucose monitoring apparatus is configured to determine a glucose test result from at least one test strip of the plurality of test strips; where the glucose data is delivered to at least one third party user by use of a graphical user interface caused to be displayed by the software. In some embodiments, the at least one third party user is a medical professional.

While a number of embodiments of the present invention have been described, it is understood that these embodiments are illustrative only, and not restrictive, and that many modifications may become apparent to those of ordinary skill in the art. Further still, the various steps may be carried out in any desired order (and any desired steps may be added and/or any desired steps may be eliminated).

1-12. (canceled)

13. A diabetes management system, comprising:
at least one server functioning in a communications cloud to provide cloud based diabetes management, the server having software stored on a non-transient computer readable medium;
wherein, upon execution of the software, the at least one server is at least configured to receive test data from a plurality of testing apparatuses, wherein each apparatus comprises:
a connecting plug configured to mate with an audio jack phone port on a portable electronic device, wherein the connecting plug is configured to receive power for the apparatus from the audio jack phone port, and wherein the connecting plug is configured to receive data from the audio jack phone port, and wherein the connecting is configured to transmit data from the glucose monitoring apparatus to the audio jack phone port; and
wherein the apparatus is configured to determine a test result and to deliver a test result to at least one third party user of a connectable device, by use of a graphical user interface caused to be displayed by the software.

14. The system of claim 13, wherein the communications cloud includes one or more cloud services.

15. The system of claim 14, wherein the cloud services are selected from the group including SaaS services, social features, CRM, Email, Virtual desktop, thin client, and terminal emulator.

16. The system of claim 13, wherein the communications cloud includes a Web Frontend.

17. The system of claim 16, wherein the Web Frontend provides PaaS services selected from the group including execution runtime, database, web server, development tools

18. The system of claim 16, wherein the communications cloud includes cloud infrastructure one or more system related databases.

19. The system of claim 13, wherein the communications cloud includes cloud infrastructure services selected from the group including virtual machines, servers, storage, load balances, and networking features.

20. The system of claim 13, wherein the software is adapted to enable recording, saving, tracking, analyzing, managing, and/or sharing of at least a portion of a user's diabetes-related data.

21. The system of claim 13, wherein the software is adapted to synchronize a user's diabetes-related data.

22. The system of claim 13, wherein the testing apparatus is a dongle.

23. The system of claim 13, wherein the portable electronic device has location tracking functionality.

24. The system of claim 13, wherein the portable electronic device has NFC communication capability, adapted to allow plurality of NFC-enable devices within close proximity to conduct peer-to-peer communication with each other.

25. The system of claim 13, wherein the at least one third party user is selected from the group consisting of a medical professional, healthcare professional, caretaker, caregiver and anyone else that can utilize the data provided by the testing apparatus to treat a user.

26. The system of claim 13, wherein the testing apparatus is configured to provide test results for self-testing outside a body.

27. A method for supporting diabetes management by a third party comprising:
receiving blood measurement test results by a server functioning in a communications cloud, the test results being generated by a diabetes patient's mobile device via a testing apparatus connectable to the mobile device's audio port;
analyzing the test results by software stored on a non-transient computer readable medium on the server; and
delivering the test results to the third party.

28. A cloud based health management platform, comprising:
a blood measurement device enabled to communicatively connect to server functioning in a communications cloud, to facilitate data capture and analysis designed to support a user's health management,
wherein the blood measurement device acquires test results via a testing apparatus connectable to an audio port on a mobile electronic device.

29. The health management platform of claim 28, further comprising an application configured to be used through software on the mobile device.

30. The health management platform of claim 28, further comprising a sharing functionality to enable sharing of health management data to selected third party users.
